(19) United States
(12) Patent Application Publication
(10) Pub. No.: US 2006/0171977 A1
(43) Pub. Date: Aug. 3, 2006

(54) Methods for the identification of fungicidally active compounds based on thymidylate kinase

(76) Inventors: Dirk Nennstiel, Lyon (FR); Verena Vollenbroich, Mulheim (DE); Peter Schreier, Koln (DE)

Correspondence Address: BAYER CROPSCIENCE LP;Patent Department, 100 BAYER ROAD, PITTSBURGH, PA, 15205-9741, US

(21) Appl. No.: 11/168,029

(22) Filed: Jun. 28, 2005

(30) Foreign Application Priority Data
Jul. 1, 2004 (DE) 102004031901.4
Jul. 7, 2004 (DE) 102004032872.2

(51) Int. Cl. A01N 25/00; C12Q 1/48; C12Q 1/18

(52) U.S. Cl. 424/405; 435/15; 435/32

(57) ABSTRACT

The invention is related to a method for the identification of fungicides, the use of thymidylate kinases for the identification of fungicides, the use of thymidylate kinase inhibitors as fungicides and methods for combating plant pathogenic fungi.

The invention is related to a method for the identification of fungicides, the use of polypeptides with thymidylate kinase enzymatic activity for the identification of fungicides, and the use of thymidylate kinase inhibitors as fungicides.

Undesired fungal growth, which leads to considerable damage in agriculture every year, can be controlled with the use of fungicides. The demands on fungicides, with respect to their efficacy, costs and most of all their environmental tolerance, have continually increased. Therefore, there is a need for new substances or substance classes that can be developed into effective and environmentally tolerable new fungicides. It is normally customary to search for such new chemical leads using greenhouse tests. However, such tests are labour-intensive and expensive. The number of substances that can be tested in the greenhouse is limited as well. An alternative to such tests is the use of so-called high throughput procedures (HTS=high throughput screening). Using HTS, a large quantity of individual substances are tested with regard to their effect on cells, individual gene products or genes in an automatic procedure. If certain substances are shown to have an effect, they can be analysed in conventional screening procedures and further developed if applicable.

Thymidylate kinase (EC 2.7.4.9), also known as thymidine monophosphate kinase, thymidylate monophosphate kinase, deoxythymidine 5′-monophosphate kinase, TMPK catalyses the reaction of ATP with thymidine 5′-monophosphate (TMP) to form thymidine 5′-diphosphate (TDP) (phosphotransferase reaction, see FIG. 1).

The reaction catalysed by thymidylate kinase represents an essential step in the preparation of the initial steps for DNA synthesis (Baranowska et al., 1990; Newlon and Fangman, 1975; Schild and Byers, 1978; Sclafani and Fangman, 1984; Zamb and Roth, 1977).

Genes for the thymidylate kinase were cloned from the fungi Saccharomyces cerevisiae (Swissprot Accession No.: P00572) and Schizosaccharomyces pombe (Swissprot Accession No.: P36590) and could also be identified in the genomes of additional fungi (e.g. Neurospora crassa EAA29458) and plant pathogenic fungi (e.g. Magnaporthe grisea MG09457.1). In addition, the thymidylate kinase was also extracted from numerous other organisms, e.g. from Homo sapiens (Swissprot: Accession No.: P23919), Arabidopsis thaliana (Fragment Swissprot: Accession No.: 081650) or Streptococcus pneumoniae (Swissprot: Accession No.: Q8DQ58).

The sequence similarities are significant within the eucaryotic classes (40-50% identity and 50-60% similarity), whereas the sequence identity with respect to the bacterial enzymes is less significant (20-25% identity and 40-50% similarity), see FIG. 2.

Of the eucaryotic TMPK's, the enzymes from yeast (Lavie et al., 1998; Lavie et al., 1997) and the human (Ostermann et al., 2000; Ostermann et al., 2003) have been heterologously expressed and crystallised in E. coli. However, to date enzyme characterisations have only been done with the purified enzyme from e.g. S. cerevisiae (Jong and Campbell, 1984) or N. crassa (Rossi and Wodward, 1975).

The object of the present invention was to identify new points of attack of fungicides in fungi, particularly in pythopathogenic fungi and to access a method with which inhibitors of such a point of attack or polypeptide can be identified and tested for their fungicidal properties. This was achieved by isolating nucleic acids coding for thymidylate kinase from a pythopathogenic fungus, extracting the thus coded polypeptide and making a method available, with which inhibitors of this enzyme can be determined. The inhibitors identified with this method can be employed against fungi in vivo.

### DESCRIPTION OF FIGURES

FIG. 1: Diagram of the reaction catalysed by the thymidylate kinase. The thymidylate kinase catalyses the reaction of deoxythymidine 5′-phosphate (dTMP) with adenosine 5′-triphosphate (ATP) to form deoxythymidine 5′-diphosphate (dTDP) and adenosine 5′-diphosphate (ADP). One ATP molecule and one dTMP molecule are consumed in the reaction.

FIG. 2: Sequential alignment of thymidylate kinases from Magnaporte grisea (M. grisea), Candida albicans (C. albicans), Neurospora crassa (N. crassa), Schizosaccharomyces pombe (S. pombe), Saccharomyces cerevisiae (S. cerevisiae), Homo sapiens (H. sapiens), Mus musculus (M. musculus) and Streptomyces pneumoniae (S. pneumoniae). Grey shading indicates the area with good conservation.

FIG. 3: SDS gel for checking the heterologous expression of thymidylate kinase in E. coli BL21(DE3)pLysS (Novagen). The super-expressed His₆ fusion protein has a size of 27.3 kDa. For each one, a marker (M) was applied externally. Lanes 3 and 4: purified thymidylate kinase; lane 1: cytoplasmic fraction of super-expressed thymidylate kinase 24 hours after induction with IPTG; lanes 5 and 6: washing fractions after applying the cytoplasmic fraction to the metal chelate sepharose column.

FIG. 4: Graphical illustration of the kinetics of conversion of thymidylate and ATP by various concentration of thymidylate kinase in the assay. In an assay volume of 50 μl, 500 μM thymidylate, 500 μM ATP, 300 μM NADH, 400 μM PEP (phosphoenol pyruvate), 0.1 U pyruvate kinase and 0.1 U lactate dehydrogenase were employed. The applied protein concentrations of thymidylate kinase are shown in the figure. The conversion occurred on the basis of a decrease in absorption at 340 nm due to the reaction of the released ADP with PEP and NADH to form lactate and NAD.

FIG. 5: Lineweaver-Burk plot for determination of the K_{M} value of dTMP (A) and ATP (B). The measured values are illustrated according to Lineweaver and Burk, i.e. 1/Vₒ=1/Vₘₐₓ+1/S×(K_{M}/Vₘₐₓ), where Vₒ is the original reaction speed, Vₘₐₓ is the maximum attainable reaction speed and S is the substrate concentration. Vₘₐₓ and K_{M} can then be calculated as the intercepts on the vertical axis and horizontal axis, 1/V and 1/K_{M}. The K_{M} value for dTMP is 0.17 mM μM, the K_{M} value for ATP is 0.16 mM μM.

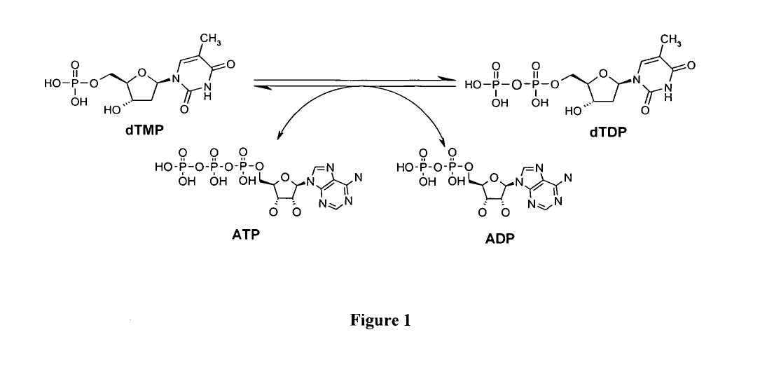

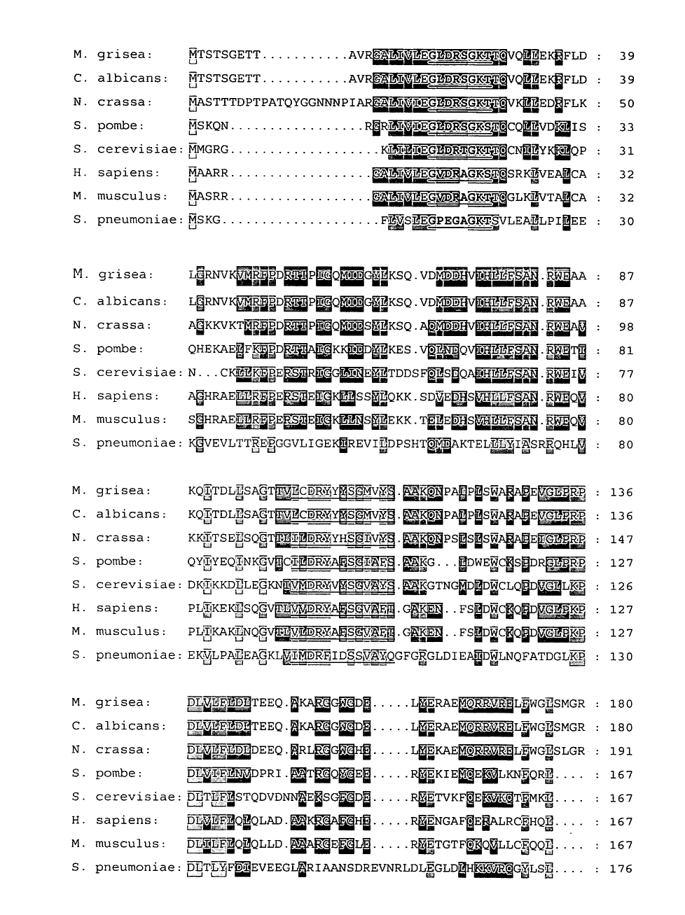

### DEFINITIONS

The term “homology” or “identity” is to be understood to mean the number of amino acids agreeing (identity) with other proteins, expressed as a percentage. The identity is preferably calculated by comparing a given sequence to other proteins with the aid of computer programs. If sequences, which are compared with one another, exhibit different lengths, the identity is calculated in a way that the number of amino acids that the shorter sequence has in common with the longer sequence determines the identity percentage. The identity can normally be calculated using known and publicly available software programs such as e.g. ClustalW (Thompson et al., Nucleic Acids Research 22 (1994), 4673-4680). ClustalW is made publicly available by Julie Thompson (Thompson@EMBL-Heidelberg.DE) and Toby Gibson (Gibson@EMBL-Heidelberg.DE), European Molecular Biology Laboratory, Meyerhofstrasse 1, D 69117 Heidelberg, Germany, for example. ClustalW can also be downloaded from various Internet sites, including the IGBMC (Institut de Génétique et de Biologie Molëculaire et Cellulaire, B.P.163, 67404 Illkirch Cedex, France; ftp://ftp-igbmc.u-strasbg.fr/pub/) and the EBI (ftp://ftp.ebi.ac.uk/pub/software/), as well as from all mirrored Internet sites of the EBI (European Bioinformatics Institute, Wellcome Trust Genome Campus, Hinxton, Cambridge CB10, 1SD, UK). If version 1.8 of the ClustalW application is used to determine the identity between a given reference protein and other proteins, for example, the following parameters should be set: KTUPLE=1, TOPDIAG=5, WINDOW=5, PAIRGAP=3, GAPOPEN=10, GAPEXTEND=0.05, GAPDIST=8, MAXDIV=40, MATRIX=GONNET, ENDGAPS(OFF), NOPGAP, NOHGAP. One possibility for finding similar sequences is the execution of sequence database queries. This means that one or more sequences are entered as so-called queries. The query sequence is then compared with sequences contained in the selected databases using statistical software. Such database queries (“blast searches”) are known to the person skilled in the art and can be performed by various providers. If such a database query is executed at the NCBI (national Center for Biotechnology Information, http://www.ncbi.nlm.nih.gov/), for example, the default settings indicated for the corresponding comparative query should be used. For protein sequence comparisons (“blastp”), these are the following settings: Limit entrez=not selected; Filter=low complexity selected; Expect value 10; word size=3; Matrix=BLOSUM62; Gap costs: Existence=11, Extension=1. The result of such a query also indicates the identity percentage between the query sequence and the similar sequences found n the databases, in addition to other parameters. Therefore, in conjunction with the present invention a protein according to the invention should be understood to mean those proteins that exhibit an identity of at least 70%, preferably at least 75%, particularly preferably at least 80%, more preferably at least 85%, and especially at least 90%, when one of the aforementioned methods is used for determining the identity.

The expression “complete thymidylate kinase” as used in this text describes a thymidylate kinase, which is coded from a complete coding region of a transcription unit beginning with the ATG start codon and including all exon regions containing information of the gene coding for thymidylate kinase occurring in the original organism, as well as the necessary signals for correct termination of the transcription.

The expression “biological” or “enzymatic” activity of a thymidylate kinase” as used in this text refers to the ability of a polypeptide to catalyse the aforementioned reaction, i.e. the conversion of dTMP and ATP into dTDP.

The expression “active fragment” as used in this text describes no longer complete nucleic acids coding for thymidylate kinase that still code for polypeptides with the enzymatic activity of a thymidylate kinase, however, and that can catalyse the characteristic reaction for thymidylate kinase as described above. Such fragments are shorter than the previously described complete fragments of nucleic acids coding for thymidylate kinase. Nucleic acids can either be removed from the 3′- and/or 5′-endens of the sequence; however, the parts of the sequence can also be deleted, which do not considerably affect the biological activity of the thymidylate kinase. Decreased or possibly also increased activity, which still allows characterisation or use of the resulting thymidylate kinase fragments, however, is understood to be sufficient in the sense of the expression used here. The expression “active fragment” can also refer to the amino acid sequence of the thymidylate kinase and, similarly to the explanations above, applies to those polypeptides that no longer contain certain sections in comparison to the complete sequence defined above, however in which the biological activity of the enzyme is not considerably affected. The fragments may be of different lengths.

The term “thymidylate kinase inhibition test” or “inhibition test” as used in this text refers to a method or a test that allows the recognition of inhibition of the enzymatic activity of a polypeptide with the activity of a thymidylate kinase by one or more chemical compounds (candidate compound(s)), whereby the chemical compound can be identified as an inhibitor of the thymidylate kinase.

The expression “gene” as used in this text is the designation for a segment from the genome of a cell, which is responsible for the synthesis of a polypeptide chain.

The expression “fungicide” or “fungicidal” as used in this text refers to chemical compounds that are suitable for combating fungi, particularly plant pathogenic fungi. Such plant pathogenic fungi are listed below, however the list is not all-inclusive:

Plasmodiophoromycetes, Oomycetes, Chytridiomycetes, Zygomycetes, Ascomycetes, Basidiomycetes and Deuteromycetes, e.g.

Pythium species, for example Pythium ultimum, Phytophthora species, for example Phytophthora infestans, Pseudoperonospora species, for example Pseudoperonospora humuli or Pseudoperonospora cubensis, Plasmopara species, for example Plasmopara viticola, Bremia species, for example Bremia lactucae, Peronospora species, for example Peronospora pisi or P. brassicae, Erysiphe species, for example Erysiphe graminis, Sphaerotheca species, for example Sphaerotheca fuliginea, Podosphaera species, for example Podosphaera leucotricha, Venturia species, for example Venturia inaequalis, Pyrenophora species, for example Pyrenophora teres or P. graminea (conidial form: Drechslera, syn: Helminthosporium), Cochliobolus species, for example Cochliobolus sativus (conidial form: Drechslera, syn: Helminthosporium), Uromyces species, for example Uromyces appendiculatus, Puccinia species, for example Puccinia recondita, Sclerotinia species, for example Sclerotinia sclerotiorum, Tilletia species, for example Tilletia caries; Ustilago species, for example Ustilago nuda or Ustilago avenae, Pellicularia species, for example Pellicularia sasakii, Pyricularia species, for example Pyricularia oryzae, Fusarium species, for example Fusarium culmorum, Botrytis species, Septoria species, for example Septoria nodorum, Leptosphaeria species, for example Leptosphaeria nodorum, Cercospora species, for example Cercospora canescens, Alternaria species, for example Alternaria brassicae or Pseudocercosporella species, for example Pseudocercosporella herpotrichoides.

Magnaporthe grisea, Cochliobulus heterostrophus, Nectria hematococcus and Phytophtora species are of particular interest, for example.

Fungicidally active compounds that are found with the aid of the thymidylate kinase according to the invention, can also be interacted with thymidylate kinases from human pathogenic fungi species, however, whereby the interaction with various thymidylate kinases occurring in these fungi does not always have to be of equal strength.

To this end, the following human pathogenic fungi are of particularly interest, which can cause the diseases listed below, among others:

Dermatophytes, such as e.g. Trichophyton spec., Microsporum spec., Epidermophyton floccosum or Keratomyces ajelloi, which cause Athlete's foot (Tinea pedis), for example,

Yeasts, such as e.g. Candida albicans cause candida oesophagitis and dermatitis, Candida glabrata, Candida krusei or Cryptococcus neoformans, which can cause pulmonary cryptococcosis and torulosis, for example,

Moulds, such as e.g. Aspergillus fumigatus, A. flavus, A. niger, which cause bronchiopulmonary aspergillosis or fungal sepsis, for example, Mucor spec., Absidia spec., or Rhizopus spec., which cause zygomycosis (intravascular mycosis) for example, Rhinosporidium seeberi, which causes chronic granulomatous pharyngits and tracheitis, for example, Madurella myzetomatis, which causes subcutaneous myzetomas, for example, Histoplasma capsulatum, which causes reticuloendothelial cytomycosis and Darling's disease, for example, Coccidioides immitis, which causes pulmonary coccidioidomycosis and sepsis, for example, Paracoccidioides brasiliensis, which causes Brasilian blastomycosis, for example, Blastomyces dermatitidis, which causes disease and North American blastomycosis, for example, Loboa loboi, which causes Keloid blastomycosis and Lobo's disease, for example, and Sporothrix schenckii, which causes sporotrichosis (granulomatous dermal mycosis), for example.

In the following text, the terms “fungicidal” and “fungicide”, “antifungal” and “antimycotic” as well as the terms should be used in the customary sense, i.e. related to plant pathogenic fungi. Fungicidally-active compounds that are found with the aid of a thymidylate kinase extracted from a certain fungus, e.g. S. cerevisiae, can thus also be interacted with a thymidylate kinase from numerous other fungal species, even with plant pathogenic fungi, whereby the interaction with the various thymidylate kinases occurring in these fungi does not always have to be of equal strength. This, among other things, explains the observed selectivity of the active substances on this enzyme.

The expression “agonist” as used in this text refers to a molecule that accelerates or strengthens the activity of the thymidylate kinase.

The expression “antagonist” as used in this text refers to a molecule that decelerates or prevents the activity of the thymidylate kinase.

The expression “modulator” as used in this text represents the higher-level term encompassing agonists and antagonists. Modulators can be small organic chemical molecules, peptides or antibodies that bond to the polypeptides according to the invention or influence their activity. Furthermore, modulators can be small organic chemical molecules, peptides or antibodies that bond to a molecule, which then bonds to the polypeptides according to the invention, and influences their biological activity in this manner. Modulators can represent natural substrates or structural or functional copies of them. The expression “modulator” as used in this text preferably means those molecules that do not represent the natural substrates or ligands, however.

### DESCRIPTION OF THE INVENTION

Despite extensive research on thymidylate kinase, it was previously unknown that thymidylate kinase can be a target protein (a so-called “target”) of fungicidally active substances in phytopathogenic fungi.

No fungicidal action has been described for the known inhibitors of the viral thymidylate kinase (see e.g. Bone et al., 1986; Davies et al., 1988; Haouz et al., 2003; Manallack et al., 2002).

In the present invention, it was shown for the first time that thymidylate kinase also represents an important enzyme for fungi and therefore is suitable for use as a target protein in the search for new and improved fungicidally active compounds, to a certain extent.

Within the framework of the present invention, it could be shown with the aid of knock-out experiments (Example 1), that the thymidylate kinase can be a point of attack or “target” for fungicidally-active substances, which can could lead to the inhibition of the thymidylate kinase and thus to damage to or extermination of the fungus. It could also be shown that the thymidylate kinase enzyme can be used for the identification of modulators or inhibitors or its enzymatic activity in test procedures, which is not always the case with various theoretically interesting targets that are already known to be essential for an organism, for example. Finally, it could also be shown that inhibitors of the thymidylate kinase, which could be identified in such procedures, can be used as fungicides.

Within the framework of the present invention, a method was developed that is suitable for determining the activity of the thymidylate kinase as well as the inhibition of this activity in a so-called inhibition test and for identifying inhibitors of the enzyme in this manner, e.g. in HTS and UHTS procedures. The compounds identified with the aid of the method according to the invention can be tested on fungi in in vivo tests.

Within the framework if the present invention, it was thus found that the thymidylate kinase can also be inhibited by compounds in vivo and a fungal organism can be damaged and killed off by treating it with these active compounds. The inhibitors of a fungal thymidylate kinase can thus be utilised as fungicides, particularly in the area of plant protection or as antimycotics in pharmaceutical indications. In the present invention, it is shown, for example, that inhibition of the thymidylate kinase with one of the substances identified in a method according to the invention, leads to extermination or growth inhibition of the fungi treated in synthetic media or on the plant.

The thymidylate kinase can be extracted in a simple manner from fungi such as S. cerevisiae, for example. The gene can be recombinantly expressed in Escherichia coli and an enzyme preparation can be produced from E. coli cells for example, in order to produce the thymidylate kinase from yeast (Example 2).

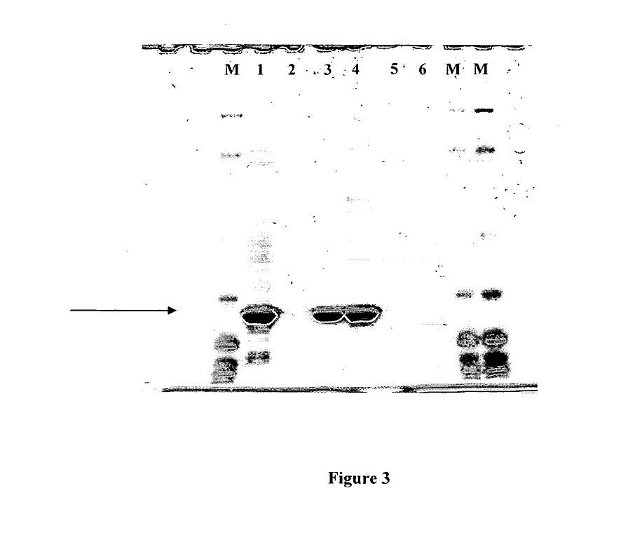

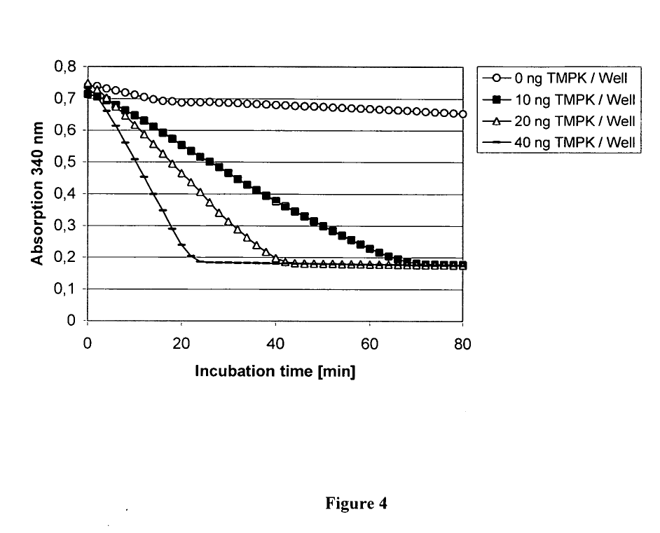

Thus, the expression of the CDC8 polypeptide from yeast coded by cdc8 (Jong and Campbell, 1984; SWISS-PROT Accession Number: P00572) of the related ORF from genomic DNA was amplified with respect to gene-specific primers according to methods known to the person skilled in the art. The corresponding DNA was cloned into the pENTR/D-TOPO vector (Invitrogen Corporation, Carlsbad, Calif., USA) and transferred into the pDest17 vector (Invitrogen) using specific recombination (Gateway-Technology Invitrogen). The resulting pDest17-cdc8 plasmid contains the complete coded sequence of cdc8 in an N-terminal fusion with a His6 tag from the vector. The CDC8 fusion protein has a calculated mass of 27 kDa (see Example 2 and FIG. 3).

The pDEST17-cdc8 plasmid was then used for recombinant expression of CDC8 in E. coli BL21(DE3)pLysS (Novagen Inc.) cells (see Example 2).

As already explained above, the present invention is not limited to the use of thymidylate kinase from yeast. In a similar manner, which is also known to the person skilled in the art, polypeptides with the activity of a thymidylate kinase can be extracted from other fungi, preferably from plant pathogenic fungi, which can then be utilised in a method according to the invention. The thymidylate kinase from S. cerevisiae is preferably used, for example.

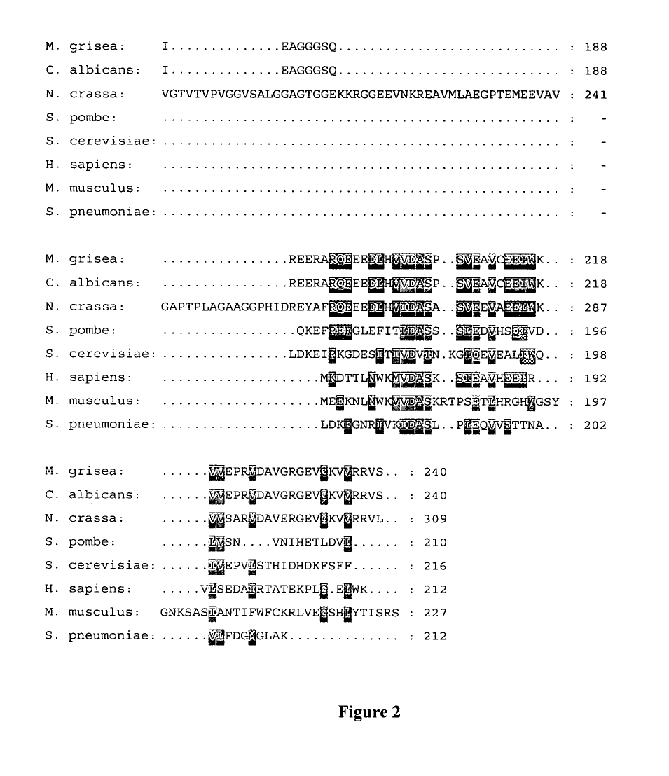

Thymidylate kinases are split into homologous regions, on the basis of which they can be identified. A motif occurring in many ATP-bonding enzymes is typical for thymidylate kinase. This preserved motif has to do with a glycine-rich region, which typically forms a flexible loop between a beta-sheet and an alpha helix. This loop interacts with one of the phosphate groups of the nucleotide. This sequence motif is generally designated as the “P-loop” and corresponds to the following sequence, which is underlined in FIG. 2:
[AG]-x(4)-G-K-[ST]

Thymidylate kinases with the sequence motif G-L-D-R-x-G-K-T are preferably used.

It is also possible to indicate another motif typical for thymidylate kinases, which enables identification of this enzyme, in addition to the enzyme test (Hofmann K., Bucher P., Falquet L., Bairoch A. (1999) “The PROSITFE database, its status in 1999”. Nucleic Acids Res. 27, 215). It can be indicated as follows:
[LIV]-[LIVMGSTC]-[DET]-[RH]-[FYHCS]-x(2)-S-[GSTNP]-x-[AVC]-[FY]-[STANQ].

Thymidylate kinases with the sequence motif [LIV]-[LIVMGSTC]-D-R-Y-x(2)-S-G-x-[AV]-[FY]-S are preferably used. The motif is underlined in FIG. 2.

PROSITE enables one to attribute a function to polypeptides and thus recognise thioredoxine reductases as such. When indicating the Prosite motif, a “one-letter code” is used. The symbol “x” stands for a position, in which every amino acid is accepted. Variable positions, in which various specific amino acids are accepted, are indicated in brackets “[ . . . ]”, in which the possible amino acids in this position are listed. Amino acids, which are not accepted in a certain position, are in curly brackets “{ . . . }”. A dash “-” divides the individual elements or positions of the motif. If a certain position is repeated several times consecutively, e.g. “x”, this can be indicated by showing the number of repetitions in parentheses, e.g. “x (3)”, which stands for “x-x-x”.

A Prosite motif thus indicates the components of a consensus sequence, as well as spaces between the amino acids involved and is therefore typical for a certain enzyme class. On the basis of this motif, by using known nucleic acids coding for thymidylate kinases, additional polypeptides from (plant pathogenic) fungi can be identified and attributed that belong to the same class as the polypeptide according to the invention and hence can also be used in the manner indicated by the invention.

In the case of the thymidylate kinase from S. cerevisiae, the same motif is also present in S. pombe, M. grisea, C. albicans and N. crassa (see FIG. 2).

The abovementioned Prosite motif and the specific consensus sequence are typical for the polypeptides according to the invention, which can be structurally defined according to this consensus sequence, and which are therefore uniquely identifiable.

Therefore, another subject of the present invention are polypeptides from plant pathogenic fungi with the enzymatic activity of a thymidylate kinase, which includes the previously mentioned Prosite motif [LIV]-[LIVMGSTC]-[DET]-[RH]-[FYHCS]-x(2)-S-[GSTNP]-x-[AVC]-[FY]-[STANQ] and the P-loops motif [AG]-x(4)-G-K-[ST].

It is possible for the person skilled in the art to obtain and identify additional nucleic acids coding for thymidylate kinases from other (plant pathogenic) fungi, e.g. by suing PCR. Such nucleic acids and their use in methods for the identification of fungicidally active compounds are considered to be included in the present invention.

The expression “polypeptide” as used in this text refers to both short amino acid chains that are usually designated as peptides, oligopeptides or oligomers and to longer amino acid chains, which are usually designated as proteins. It includes amino acid chains that can be modified either by natural processes, such as posttranslational processing or by chemical procedures, which are state-of-the-art. Such modifications can occur in various locations and several times in a polypeptide, for example on the peptide backbone, on the amino acid secondary chain, on the amino terminus and/or on the carboxy terminus. For example, they include acetylations, acylations, ADP ribosylations, amidations, covalent links with flavins, haem segments, nucleotides or nucleotide derivatives, lipids or lipid derivatives or phophatidylinositol, cyclisations, disulphide bridge formations, demethylations, cystine formations, formylations, gamma-carboxylations, glycosylations, hydroxylations, iodinations, methylations, myristoylations, oxidations, proteolytic processes, phosphorylations, selenoylations and tRNA-mediated additions of amino acids.

The polypeptides may according to the invention be used in the form of “mature” proteins or as parts of larger proteins, e.g. as fusion proteins. They may furthermore have secretions or “leader” sequences, pro-sequences, sequences that make simple purification possible, such as multiple histidine residues, or additional stabilising amino acids. The proteins according to the invention can also occur as they do naturally in their original organism, from which they can be directly extracted, for example. In the method according to the invention, active fragments of a thymidylate kinase can be employed as well as long as they allow determination of the enzymatic activity of the polypeptide or their inhibition by a candidate compound.

The polypeptides utilised in the method according to the invention can exhibit deletions or amino acid substitutions in comparison to the corresponding regions of naturally occurring thymidylate kinases as long as they still exhibit at least the biological activity of one complete thymidylate kinase. Conservative substitutions are preferred. Such conservative substitutions include variations in which an amino acid is substituted by another amino acid from the following group:
1. Small aliphatic, non-polar or slightly polar moieties: Ala, Ser, Thr, Pro and Gly;
2. Polar, negatively charged moieties and their amides: Asp, Asn, Glu and Gln;
3. Polar, positively charged moieties: His, Arg and Lys;
4. Large aliphatic, non-polar moieties: Met, Leu, Ile, Val and Cys; and
5. Aromatic moieties: Phe, Tyr and Trp.

A possible method for purification of the thymidylate kinase is based on preparative electrophoresis, FPLC, HPLC (e.g. using gel filtration, reverse phase or slightly hydrophobic columns), gel filtration, differential precipitation, ion-exchange chromatography or affinity chromatography (see Example 2).

A rapid method for isolating thymidylate kinases according to the invention, which are synthesized by host cells, starts with the expression of a fusion protein, where the fusion partner can be affinity-purified in a simple manner. The fusion partner may be, for example, a His6 tag (see Example 2). The fusion protein can then be purified on Ni-NTA-agarose. The fusion partner can be removed by partial proteolytic cleavage, for example at linkers between the fusion partner and the polypeptide according to the invention, which is to be purified. The linker can be designed so that it includes target amino acids, such as arginine and lysine residues, which define sites for cleavage by trypsin. Such linkers can be generated by employing standard cloning methods using oligonucleotides.

Further possible purification methods are based once again on preparative electrophoresis, FPLC, HPLC (e.g. using gel filtration, reverse phase or slightly hydrophobic columns), gel filtration, differential precipitation, ion exchange chromatography and affinity chromatography.

The terms “isolation or purification” as used in this text mean that the polypeptides according to the invention are separated from other proteins or other macromolecules of the cell or the tissue. A composition according to the invention containing the polypeptides is preferably enriched at least 10-fold and particularly preferably at least 100-fold, in terms of the protein content, compared with a preparation from the host cells.

The polypeptides according to the invention can also be affinity-purified without fusion partners with the aid of antibodies, which bind to the polypeptides.

The method for the production of polypeptides with the activity of a thymidylate kinase, such as e.g. the CDC8 polypeptide, is characterised in
(a) the cultivation of a host cell containing at least one expressible nucleic acid sequence coding for a polypeptide from fungi with the biological activity of a thymidylate kinase under conditions that guarantee the expression of this nucleic acid, or
(b) the expression of an expressible nucleic acid sequence coding for a polypeptide from fungi with the biological activity of a thymidylate kinase in an in vitro system, and
(c) the extraction of the polypeptide from the cell, the culture medium or the in vitro system.

The thus obtained cells containing the polypeptide according to the invention or the thus obtained purified polypeptide are suitable to be used in procedures for the identification of modulators or inhibitors of the thymidylate kinase.

A further subject of the present invention is the use of polypeptides from fungi, which express the enzymatic activity of a thymidylate kinase class, in procedures for the identification of inhibitors of polypeptides with the activity of a thymidylate kinase, whereby the inhibitors of the thymidylate kinase can be utilised as fungicides. The thymidylate kinase from S. cerevisiae is particularly preferably used.

Fungicidally active compounds that are found with the aid of a thymidylate kinase from a certain fungal species can also be interacted with thymidylate kinases from other fungal species, whereby the interaction with the various thymidylate kinases occurring in these fungi does not always have to be equally strong. This, among other things, explains the selectivity of active substances. The use of active compounds that were found with a thymidylate kinase of a particular fungal species as fungicides for other fungal species can be linked to the fact that thymidylate kinases from various fungal species are very similar and exhibit a pronounced homology in large areas. In FIG. 2, it is clear that such a homology exists over considerable sequence segments among S. cerevisiae, N. crassa and S. pombe and therefore the function of the substances found with the aid of the thymidylate kinase from yeast is not limited to S. cerevisiae. In procedures for the identification of fungicides, therefore, polypeptides with the enzymatic activity of a thymidylate kinase are preferably used, which exhibit a consensus sequence according to FIG. 2.

Therefore, another subject of the present invention is a method of identification of fungicides by testing potential inhibitors and modulators of the enzymatic activity of thymidylate kinase (test compound) in a thymidylate kinase inhibition test (Example 3).

Methods that are suitable for identifying modulators, particularly inhibitors or antagonists of the polypeptides according to the invention, are generally based on determining the activity or the biological function of the polypeptide. For this purpose, both methods involving entire cells (in vivo methods) and methods concentrating on the use of the polypeptide isolated from the cells come into consideration, whereby the polypeptide can occur in a purified or partially purified form or as a raw extract. These cell-free in vitro methods can also be used as in vivo methods on a laboratory scale, or preferably in HTS or UHTS methods. After completion of the in vivo or in vitro identification of modulators of the polypeptide, tests can be performed on fungal cultures in order to verify the fungicidal activity of the identified compounds.

Many test systems that have the goal of testing compounds and natural extracts are directed towards high throughput quantities, in order to maximise the number of substances examined in a given time period. Test systems based on cell-free methods require purified or semi-purified protein. They are suitable for an “initial” test, which is mainly aimed at detecting a possible influence of a substance on the target protein. If such an initial test occurs and one or more compounds, extracts, etc. are found, the activity of these compounds can be more precisely studied in the laboratory. Thus, as a first step, the inhibition or activation of the polypeptide according to the invention can be tested once again in vitro, in order to subsequently test the efficacy of the compound in the target organism, in this case on one or more plant pathogenic fungi. The compound can then be used as a starting point for continuing the search and development of fungicidal compounds, which are based on the original structure, but which are optimised with respect to the efficacy, toxicity or selectivity, for example.

For the purpose of finding modulators, a synthetic reaction mix (e.g. products of in vitro transcription), a cellular component such as a membrane, compartment or any other preparation that contains polypeptides according to the invention can be incubated together with a tagged substrate or ligand of the polypeptide, if applicable, in the presence and absence of a test compound (or a mixture of test compounds), which can be an antagonist. The ability of the test compound to inhibit the thymidylate kinase activity is recognisable, for example, by decreased conversion of the tagged substrate (if applicable).

Detection of biological activity of the polypeptide according to the invention can be improved by a so-called reporter system. Reporter systems, in this respect, include but are not limited to colourimetrically or fluorimetrically detectable substrates, which are converted into a product or a reporter gene that responds to changes in the activity or expression of the polypeptide according to the invention, or other known bonding tests.

A further example of a method, with which modulators of the polypeptides according to the invention can be identified, is a displacement test, in which the polypeptides according to the invention and a potential modulator are exposed to a molecule that is known to bind to the polypeptides according to the invention, such as a natural substrate or ligand or an imitation substrate or ligand. The polypeptides according to the invention can be tagged, e.g. fluorimetrically or colourimetrically, so that one can precisely determine the quantity of polypeptides that are bonded to a ligand or that have undergone a conversion. However, the bond can also be tracked using the tagged substrate, ligand or substrate analogue, if applicable. The effectiveness of antagonists can be measured in this manner.

Effects such as cell toxicity are generally ignored in these in vitro systems. The test systems examine both inhibitory and suppressive, as well as stimulatory effects of the substances. The efficacy of a substance can be checked using concentration-dependent test series'. Control batches without test substances or without enzymes can be used for evaluating the effects.

Another option for the identification of substances that modulate the activity of the polypeptides according to the invention is the so-called “Scintillation Proximity Assay” (SPA), see EP 015 473. This test system utilises the interaction of a polypeptide (e.g. thymidylate kinase from yeast) with a radioactively tagged ligand or substrate. The polypeptide is bonded to small spheres (microspheres) or beads, which are equipped with the scintillating molecules. Over the course of radioactive decay, the scintillating substance in the sphere is stimulated by the subatomic particles of the radioactive marker and emits a detectable photon. The testing conditions are optimised in such a way that only the particles originating in one of the ligands bonded to the polypeptide according to the invention create a signal.

The modulators being identified are preferably small organo-chemical compounds.

A method for the identification of a compound that modulates the activity of a thymidylate kinase from fungi, and that can be used as a fungicide in the area of plant protection, is therefore characterised in that
a) a polypeptide with the enzymatic activity of a thymidylate kinase, preferably from fungi, particularly preferably from plant pathogenic fungi, or a host cell containing such a polypeptide is exposed to a chemical compound or with a mixture of chemical compounds under conditions, which allow the interaction of a chemical compound with the polypeptide,
b) the activity of the polypeptide in the absence of a chemical compound is compared with the activity of the polypeptide according to the invention in the presence of a chemical compound or a mixture of chemical compounds, and
c) the chemical compound is identified that specifically modulates the activity of the polypeptide according to the invention and
d) p the fungicidal activity of the identified compound is possibly tested in vivo.

Those compounds are particularly preferably identified that specifically inhibit the activity of the polypeptide according to the invention. The term “activity” as used in this text refers to the biological activity of the polypeptide according to the invention.

The compounds identified in this manner can be utilised as fungicides for combating fungi, preferably plant pathogenic fungi. Therefore, a further subject of the present invention is a method fro the identification of fungicides, characterised in that
a) the abovementioned Steps (a) to (d) are performed,
b) the identified fungicidal compound is formulated in a suitable manner, and
c) the identified compound is allowed to interact with the fungal antagonist and/or its habitat.

A preferred method (see Example 3) makes use of the fact that an adenosine diphosphate molecule (ADP) is released during the thymidylate kinase reaction. The activity, or the increase or decrease in activity, of the polypeptide according to the invention can thus be determined via detection of the resulting ADP. The decreased or inhibited activity of the respective thymidylate kinase, which is expressed through the decreased occurrence of ADP, can be tracked by detecting the ADP resulting from the subsequent reaction of pyruvate kinase and lactate dehydrogenase. The pyruvate kinase converts phosphoenol pyruvate into pyruvate while consuming the ADP according to the following equation ADP+Phosphoenol pyruvate [symbol] ATP+Pyruvate, which is then used by the lactate dehydrogenase for oxidation of NADH to form NAD. The increased or decreased NAD-concentration, resulting from the coupled reaction, can be determined by measuring absorption or fluorescence (at 340 nm or a stimulation wavelength of 360 nm and an emission wavelength of 465 nm).

The measurement can also occur in formats suitable for HTS or UHTS assays, e.g. in microtiter plates, in which e.g. a total volume of 5 to 50 μl is prepared per batch or per well, and in which the individual components are present in the desired final concentrations (see Example 3). The compound to be tested, which potentially inhibits or activates the enzyme activity (candidate molecule), is prepared e.g. in a suitable concentration in a test buffer containing dTMP, adenosine triphosphate, phosphoenol pyruvate and the coupled auxiliary enzymes. The polypeptide according to the invention is then added to the test buffer and the reaction is started. The batch is next incubated for e.g. up to 30 minutes at a suitable temperature and the reduction in absorption is measured at 340 nm.

Another measurement is taken in a corresponding batch, however without adding a candidate molecule (test compound) and without adding thymidylate kinase (negative control). A further measurement is taken once again in the absence of a candidate molecule, but in the presence of thymidylate kinase (positive control). The negative and positive controls thus provide the comparative values to be compared with the batches in the presence of a candidate molecule.

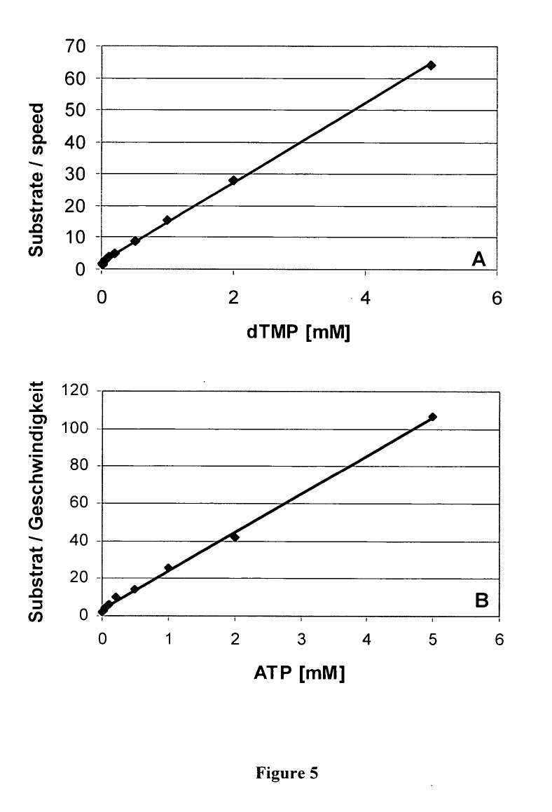

In order to provide optimal conditions for a method for the identification of thymidylate kinase inhibitors or for determination of activity of the polypeptide according to the invention, it can be advantageous to calculate the respective K_{M} value of the utilised polypeptide according to the invention. This provides information about the preferred concentration of the substrate or substrates to be used. In the case of thymidylate kinase from yeast, the K_{M} is 0.17 mM for dTMP and a K_{M} of 0.16 mM for ATP were determined (FIG. 5).

With the aid of the previously described methods, by way of example, within the framework of the present invention compounds could be identified, which inhibit the fungal thymidylate kinase and are capable of damaging (e.g. growth inhibition) or killing fungi of various species.

Of course, in addition to the methods mentioned for determining the enzymatic activity of a thymidylate kinase or determining the inhibition of this activity and for identification of fungicides, other, e.g. already known, methods or inhibition tests can also be used as long as these methods allow the determination of thymidylate kinase activity and the recognition of inhibition of this activity via a test compound.

The inhibitors of a thymidylate kinase according to the invention, which are identified with the aid of a method according to the invention, are suitable for damaging or killing fungi, preferably plant pathogenic fungi.

In order to check whether a compound identified using a method according to the invention is suitable for employment as a combatant of plant pathogenic fungi in vivo, a solution of the active substance being tested can be pipetted into microtiter plate cavities, for example. After the solvent evaporates, a medium is added to each cavity. The medium was previously mixed with a suitable concentration of spores or micelles of the fungus being tested. The resulting concentrations of active substances total, e.g. 0.1, 1, 10 and 100 ppm.

The plates are subsequently incubated on an agitator at a temperature of 22° C. until sufficient growth is detectable in the treated control.

The present invention, therefore, also relates to the use of modulators of thymidylate kinase from fungi, preferably from plant pathogenic fungi, as fungicides.

The present invention also relates to methods for combating plant pathogenic fungi, characterised in that an inhibitor of the fungal thymidylate kinase, possibly in a suitable formulation, is allowed to interact with the fungal antagonist and/or its habitat.

The present invention also relates to fungicides that were identified with the aid of a method according to the invention.

Compounds, which are identified with the aid of a method according to the invention and exhibit fungicidal activity on the basis of inhibition of the fungal thymidylate kinase, can then be used for the manufacture of fungicidal agents.

The identified active compounds can be converted into the customary formulations depending on their respective physical and/or chemical properties, such as solutions, emulsions, suspensions, powders, foams, pastes, granules, aerosols, micro encapsulations in polymeric substances and in coating compositions for seeds and ULV cool and warm-fogging formulations.

These formulations are produced in a known manner, for example by mixing the active compounds with extenders, that is, liquid solvents, liquefied gases under pressure and/or solid carriers, optionally with the use of surfactants, that is emulsifiers and/or dispersants and/or foam formers. If the extender used is water, it is also possible to employ, for example, organic solvents as auxiliary solvents. Suitable liquid solvents are essentially: aromatics such as xylene, toluene or alkylnaphthalenes, chlorinated aromatics or chlorinated aliphatic hydrocarbons such as chlorobenzenes, chloroethylenes or methylene chloride, aliphatic hydrocarbons such as cyclohexane or paraffins, for example petroleum fractions, alcohols such as butanol or glycol and their ethers and esters, ketones such as acetone, methyl ethyl ketone, methyl isobutyl ketone or cyclohexanone, strongly polar solvents such as dimethylformamide or dimethyl sulphoxide, or else water. Liquefied gaseous extenders or carriers are to be understood as meaning liquids which are gaseous at standard temperature and under atmospheric pressure, for example aerosol propellants such as halogenated hydrocarbons, or else butane, propane, nitrogen and carbon dioxide. Suitable solid carriers are: for example ground natural minerals such as kaolins, clays, talc, chalk, quartz, attapulgite, montmorillonite or diatomaceous earth, and ground synthetic minerals such as highly disperse silica, alumina and silicates. Suitable solid carriers for granules are: for example crushed and fractionated natural rocks such as calcite, marble, pumice, sepiolite and dolomite, or else synthetic granules of inorganic and organic meals, and granules of organic material such as sawdust, coconut shells, maize cobs and tobacco stalks. Suitable emulsifiers and/or foam formers are: for example non-ionic and anionic emulsifiers, such as polyoxyethylene fatty acid esters, polyoxyethylene fatty alcohol ethers, for example alkylaryl polyglycol ethers, alkylsulphonates, alkyl sulphates, arylsulphonates, or else protein hydrolysates. Suitable dispersants are: for example lignin-sulphite waste liquors and methylcellulose.

Tackifiers such as carboxymethylcellulose and natural and synthetic polymers in the form of powders, granules or lattices, such as gum arabic, polyvinyl alcohol and polyvinyl acetate, or else natural phospholipids such as cephalins and lecithins and synthetic phospholipids can be used in the formulations. Other possible additives are mineral and vegetable oils.

It is possible to use colorants such as inorganic pigments, for example iron oxide, titanium oxide and Prussian Blue, and organic dyestuffs such as alizarin dyestuffs, azo dyestuffs and metal phthalocyanine dyestuffs, and trace nutrients such as salts of iron, manganese, boron, copper, cobalt, molybdenum and zinc.

The formulations generally comprise between 0.1 and 95 percent by weight of active compound, preferably between 0.5 and 90%.

The active compounds according to the invention, as such or in their formulations, can also be used in a mixture with known fungicides, bactericides, acaricides, nematicides or insecticides, for example to widen the activity spectrum or to prevent the development of resistance. In many cases, synergistic effects are obtained, i.e. the activity of the mixture is greater than the activity of the individual components.

When employing the compounds according to the invention as fungicides, the application quantities can be varied within a large range.

All plants and plant parts can be treated according to the invention. In this context, plants are to be understood to mean all plants and plant populations, such as desired and undesired wild plants or crop plants (including naturally occurring crop plants). Crop plants can be plants that can be obtained with conventional breeding and optimisation methods or with biotechnological and recombinant methods or combinations of these methods, including transgenic plants and including plant species, which are capable, or incapable, of being protected by Plant Breeders' Rights. Plant parts should be understood as meaning all aerial and subterranean parts and organs of the plants, such as shoots, leaves, flowers and roots, examples mentioned being leaves, needles, stalks, stems, flowers, fruiting bodies, fruits and seeds, and also roots, tubers and rhizomes. The plant parts also include harvested material and vegetative and generative propagation material, for example cuttings, tubers, rhizomes, offshoots and seeds.

Treatment according to the invention of the plants and plant parts occurs directly or indirectly through interaction with their environment, habitat or storage location according to the customary treatment methods, e.g. by dipping, spraying, vaporising, atomising, scattering, brushing on and with propagation material, particularly with seeds by coating once or twice.

The following examples illustrate various aspects of the present invention and should not be considered all-inclusive.

### EXAMPLES

### Example 1

Production of cdc8 Knockout Mutants in U. maydis

Cultivation of U. maydis

The strains were cultured at 28° C. in PD, YEPS or suitable minimal media (Holliday, 1974; Tsukuda et al., 1988). The formation of dicaryotic filaments was observed after dripping the strains onto PD plate media, which contained 1% charcoal (Holliday, 1974). Pathogenicity tests were performed as described (Gillissen et al., 1992). Overnight cultures of the strains were re-suspended in a concentration of 4×10⁷ cells and young maize plants (Gaspe Flint) were injected or the suspension was dripped onto them. For every strain, at least 25 plants were infected and occurring tumours were examined after 14-21 days.

Production of the Knockout Cassette

Molecular biological standard methods were used according to Sambrook et al., 1989. In order to produce cdc8-null mutants, the 5′ and 3′ flank of the cdc8 gene were amplified using PCR. Genomic DNA of the UM518 strain served as a template. The LB2 primer with the sequence (5′-cacggcctgagtggcccggacggcgctgctgtcagttggg-3′) and p15 with the sequence (5′-gccaccatcgagtcaggaacgatgg-3′) were employed for the 5′ flank (1314 bp). The RB1 primer (5′-gtgggccatctaggccggcggtgcacagggtgacatcgtcc-3′) and p13 (5′-caccaatagcactcgacgcacgtcc-3′) were used for the 3′ flank (1238 bp). The Sfi I (a) and Sfi I (b) restrictases were introduced into the LB2 and RB1 primers. The amplicons were tied together with Sfi I and ligated with the 1884 bp large Sfi I fragment from the pBS-isolated vector (hygromycinB cassette). The 3869 bp large cdc8 knock-out cassette was amplified using PCR with the LB1 primer (5′-ggtcatccagaagggcttctcgc-3′) and RB2 primer (5′-gcttgccgtcgctggatcggagagg-3′), and was then used in the subsequent transformation (Kaemper and Schreier, 2003).

Production of Protoplasts from U. maydis

50 ml of a culture in YEPS medium was grown at 28° C. to a cell thickness of approx. 5×10⁷/ml (OD 0.6 to 1.0) and then centrifuged for 7 min. at 2500 g (Hereaus, 3500 rpm) in a 50 ml Falkon tube. The cell pellet was re-suspended in 25 ml SCS buffer (20 mM sodium citrate pH 5.8, 1.0 M sorbitol, (20 mM sodium citrate/1.0 M sorbitol and 20 mM citric acid/1.0 M sorbitol are mixed and adjusted to a pH of 5.8 using a pH meter)), once again centrifuged for 7 min. at 2500 g (3500 rpm) and the pellet was re-suspended in 2 ml SCS buffer, pH 5.8, with 2.5 mg/ml Novozyme 234. The protoplasting occurred at room temperature and was observed microscopically every 5 minutes. The protoplasts were then mixed with 10 ml SCS buffer and centrifuged at 1100 g (2300 rpm) for 10 minutes. The supernatant was discarded. The pellet was carefully re-suspended in 10 ml SCS buffer and centrifuged again. The washing procedure with SCS buffer was repeated twice; the pellet was in 10 ml STC buffer. Finally, the pellet was re-suspended in 500 μl of cold STC buffer (10 mM Tris/HCL pH 7.5, 1.0 M sorbitol, 100 mM CaCl₂) and kept on ice. Aliquots can be stored for several months at −80° C.

Transformation of U. maydis

The transformation of a diploidic U maydis strain occurred according to Schulz et al., 1990. The isolation of genomic U maydis DNA took place as described by Hoffman and Winston, 1987 and according to the Qiagen Company protocol (DNeasy-Kit).

For the transformation, a maximum of 10 μl DNA (optimally 3-5 μg) was transferred into a 2 ml Eppendorf tube, 1 μl heparin (15 μg/μl) (SIGMA H3125) was added and then 50 μl of protoplasts were set into the mixture, and the mixture was incubated on ice for 10 min. 500 μl 40% (w/w) PEG3350 (SIGMA P3640) were added to STC (sterilely filtered), carefully mixed with the protoplast suspension and incubated on ice for 15 min. The plating occurred on gradient plates (lower agar layer: 10 ml YEPS/1.5% agar/1M sorbitol with antibiotic). Shortly before plating, the lower agar layer was coated with 10 ml YEPS/1.5% agar/1M sorbitol, the protoplasts were applied and the plates were incubated for 3-4 days at 28° C.

The detection of homologous recombination in a genomic locus of cdc8 occurred using standard methods (PCR or Southern analysis) of isolated genomic DNA. This showed that the integration of the cdc8 knockout cassette into a genomic cdc8-locus had taken place and a wild-type copy of the cdc8-gene was replaced, while the second copy in this diploidic strain remained intact. The heterozygotic cdc8 mutants that resulted in this manner were subsequently used in the pathogenicity test.

Spore Analysis of U. maydis

Spores were isolated from the tumours created in the pathogenicity test. Next, the resulting spores were individually genetically and phenotypically examined. The phenotypic analysis occurred via growth trials in suitable full or minimal media (Holliday, 1974; Tsukuda et al., 1988). It was shown that no analysed sporidia grew under selective conditions. No diploidic strains were found. This was the first indication that the phenotype of the cdc8-null mutants was lethal. The genotypic analyses occurred using Southern or PCR-based analyses of the sporidia. It was found that no visable haploidic strains could be identified, in which the cdc8-gene was replaced with the cdc8 knockout cassette. From these results, it was subsequently concluded that the knockout of the cdc8-gene in Ustilago maydis leads to a lethal phenotype.

### Example 2

Cloning, Expression and Purification of cdc8 or CDC8 from Saccharomyces cerevisiae

For cloning cdc8 and the expression of such, the ORF was amplified from genomic DNA of Saccharomyces cerevisiae with respect to gene specific primers. The corresponding DNA, an amplicon of 651 bp of length, was cloned into the pENTR/D-TOPO vector (Invitrogen Corporation, Carlsbad, Calif., USA) and transferred into the pDest17 vector (Invitrogen) using specific recombination (Gateway-Technology Invitrogen). The resulting pDest17-cdc8 plasmid contains the complete coding sequence of cdc8 in an N-terminal fusion with a His6 tag from the vector. The CDC8 fusion protein has a calculated mass of 27 kDa (FIG. 3).

For heterologous expression, the pDest17-cdc8 plasmid was transformed in E. coli BL21(DE3)pLysS. A pre-culture was inoculated with the transformants in 50 ml selection medium. These cells were incubated overnight at 37° C. and then diluted to 1:15 in selection medium (LB medium with 80 μg/ml ampicillin). The induction occurred at an OD_{600 nm} of from 0.7 with 2 mM IPTG (final concentration) at 28° C. After 24 hours of induction, the cells were harvested and directly processed.

The digestion occurred via sonification in the digestive buffer (20 mM Tris, 50 mM NaCl, 40 μM TMP, 10% glycerol, 4 mM MgCl₂, pH 7.4). The cytoplasmic fraction obtained through centrifugation (20 min at 4° C., 10 000 g) was used for purification of the expressed protein. The purification occurred according to the standard instructions of the manufacturer of the 5 mL HiTrap chelating (Ni) column (Amersham Biosciences). For this purpose, the column was equilibrated with 10 column volumes (SV) of digestive buffer and washed with 7 SV of digestive buffer following the test application. Subsequently, it was washed with 10 SV washing buffer (20 mM Tris, 0.5 M NaCl, 4 mM MgCl₂, 10% glycerol, 40 μM TMP, 20 mM imidazole, pH 7.4) and pure thymidylate kinase was eluted (20 mM Tris, 50 mM NaCl, 4 mM MgCl₂, 10% glycerol, 10 μM TMP, 400 mM imidazole, pH 7.4). The purified protein was stored in elution buffer at −8⁰° C.

### Example 3

Identification of Thymidylate Kinase Modulators in 384-well MTP's in a Coupled Assay

For the identification of thymidylate kinase modulators, 384-well microtiter plates by Grenier were used.

The negative control was pipetted into the first column. This is comprised of 5 μl 5% DMSO and 25 μl buffer1 (75 mM MOPS buffer pH 7.5, 10 mM MgCl₂, 5 mM DTT, 1 mM TMP, 100 mM NaCL, 50 mM KCl). The positive control was pipetted into the second column, which is comprised of 5 μl 5% DMSO and 25 μl buffer2 (buffer1 with 1.6 μg/ml thymidylate kinase).

A test substance in a concentration of 2 μM in DMSO is prepared in the remaining columns, whereby a volume of 5 μl water was used for dilution of the substance. After adding 20 μl buffer2, 20 μl buffer3 (75 mM MOPS buffer pH 7.5, 10 mM MgCL2, 0.75 mM NADH, 1.25 mM ATP, 1.25 mM PEP, 6.25 mM DTT, 0.025% Tween 20, 125 mM NaCL, 62.5 mM KCl, 5 U/mL PK, 5 U/mL LDH) is added to all wells to start the reaction.

It was incubated at 37° C. for 40 minutes and was subsequent measured by determining the absorption at 340 nm in a SPECTRAFluor Plus from Tecan suitable for microtiter plates.

### Example 4

Detection of Fungicidal Activity in the Identified Thymidylate Kinase Inhibitors

A methanol solution, comprised of the active compound identified on the basis of one of the methods according to the invention in the desired quantity mixed with an emulsifier, is pipetted into the cavities of microtiter plates. After the solvent evaporates, 200 μl potato dextrose medium is added to each cavity. The medium was previously mixed with suitable concentrations of spores or micelles of the fungus being tested.

The resulting concentration of the emulsifier totals 300 ppm.

The plates were then incubated in an agitator at a temperature of 22° C. until sufficient growth is detectable in the untreated control. The evaluation occurs photometrically at a wavelength of 620 nm. The active compound dosage, which leads to a 50% inhibition of fungal growth with respect to the untreated control (ED₅₀), is calculated from the data measured for various concentrations.

### REFERENCED LITERATURE

1. Baranowska, H., Zaborowska, D., Jachymczyk, W. J., and Zuk, J. (1990). Role of the CDC8 gene in the repair of single strand breaks in DNA of the yeast Saccharomyces cerevisiae. In Current Genetics, pp. 175-179.
2. Bone, R., Cheng, Y. C., and Wolfenden, R. (1986). Inhibition of adenosine and thymidylate kinases by bisubstrate analogs. In Journal of Biological Chemistry (United States), pp. 16410-16413.
3. Davies, L. C., Stock, J. A., Barrie, S. E., Orr, R. M., and Harrap, K. R. (1988). Dinucleotide analogues as inhibitors of thymidine kinase, thymidylate kinase, and ribonucleotide reductase. In Journal of Medicinal Chemistry (United States), pp. 1305-1308.
4. Gillissen, B., Bergemann, J., Sandmann, C., Schroeer, B., Bolker, M., and Kahmann, R. (1992). A two-component regulatory system for self/non-self recognition in Ustilago maydis. In Cell (United States), pp. 647-657.
5. Haouz, A., Vanheusden, V., Munier-Lehmann, H., Froeyen, M., Herdewijn, P., Van Calenbergh, S., and Delarue, M. (2003). Enzymatic and Structural Analysis of Inhibitors Designed against Mycobacterium tuberculosis Thymidylate Kinase. In Journal of Biological Chemistry, pp. 4963-4971.
6. Hoffman, C. S., and Winston, F. (1987). A ten-minute DNA preparation from yeast efficiently releases autonomous plasmids for transformation of Escherichia coli. In Gene, pp. 267-272.
7. Holliday, R. (1974). Ustilago maydis. In Handbook of Genetics, R. C. King, ed. (New York, Plenum), pp. 575-595.
8. Jong, A. Y. S., and Campbell, J. L. (1984). Characterization of Saccharomyces cerevisiae thymidylate kinase, the CDC8 gene product. General properties, kinetic analysis, and subcellular localization. In Journal of Biological Chemistry, pp. 14394-14398.
9. Kaemper, J., and Schreier, P. (2003). A PCR method for the rapid construction of vectors for the generation of deletion mutants by gene knockout. In PCT Int. Appl. (WO, (Bayer Aktiengesellschaft, Germany).), pp. 51 pp.
10. Lavie, A., Konrad, M., Brundiers, R., Goody, R. S., Schlichting, I., and Reinstein, J. (1998). Crystal structure of yeast thymidylate kinase complexed with the bisubstrate inhibitor P1-(5′-adenosyl) P5-(5′-thymidyl) pentaphosphate (TP5A) at 2.0 A resolution: implications for catalysis and AZT activation. In Biochemistry (United States), pp. 3677-3686.
11. Lavie, A., Vetter, I. R., Konrad, M., Goody, R. S., Reinstein, J., and Schlichting, I. (1997). Structure of thymidylate kinase reveals the cause behind the limiting step in AZT activation. In Nature Structural Biology, pp. 601-604.
12. Manallack, D. T., Pitt, W. R., Herdewijn, P., Balzarini, J., De Clercq, E., Sanderson, M. R., Sohi, M., Wien, F., Munier-Lehmann, H., Haouz, A., and Delarue, M. (2002). Database searching for thymidine and thymidylate kinase inhibitors using three-dimensional structure-based methods. In Journal of Enzyme Inhibition and Medicinal Chemistry, pp. 167-174.
13. Newlon, C. S., and Fangman, W. L. (1975). Mitochondrial DNA synthesis in cell cycle mutants of Saccharomyces cerevisiae. In Cell (Cambridge, Mass., United States), pp. 423-428.
14. Ostermann, N., Schlichting, I., Brundiers, R., Konrad, M., Reinstein, J., Veit, T., Goody, R. S., and Lavie, A. (2000). Insights into the phosphoryl transfer mechanism of human thymidylate kinase gained from crystal structures of enzyme complexes along the reaction coordinate. In Structure (London), pp. 629-642.
15. Ostermann, N., Segura-Pena, D., Meier, C., Veit, T., Monnerjahn, C., Konrad, M., and Lavie, A. (2003). Structures of human thymidylate kinase in complexes with prodrugs: implications for the structure-based design of novel compounds. In Biochemistry (United States), pp. 2568-2577.
16. Rossi, M., and Wodward, D. O. (1975). Enzymes of deoxythymidine triphosphate biosynthesis in Neurospora crassa mitochondria. In Journal of Bacteriology, pp. 640-647.
17. Sambrook, J., Fritsch, E. F., and Maniatis, T. (1989). Molecular cloning: A laboratory manual. (Cold spring harbor, New York, Cold spring harbor laboratory press).
18. Schild, D., and Byers, B. (1978). Meiotic effects of DNA-defective cell division cycle mutations of Saccharomyces cerevisiae. In Chromosoma, pp. 109-130.
19. Schulz, B., Banuett, F., Dahl, M., Schlesinger, R., Schaefer, W., Martin, T., Herskowitz, I., and Kahmann, R. (1990). The b alleles of U. maydis, whose combinations program pathogenic development, code for polypeptides containing a homeodomain-related motif. In Cell (Cambridge, Mass., United States), pp. 295-306.
20. Sclafani, R. A., and Fangman, W. L. (1984). Yeast gene CDC8 encodes thymidylate kinase and is complemented by herpes thymidine kinase gene TK. In Proceedings of the National Academy of Sciences of the United States of America, pp. 5821-5825.
21. Tsukuda, T., Carleton, S., Fotheringham, S., and Holloman, W. K. (1988). Isolation and characterization of an autonomously replicating sequence from Ustilago maydis. In Molecular and Cellular Biology, pp. 3703-3709.
22. Zamb, T. J., and Roth, R. (1977). Role of mitotic replication genes in chromosome duplication during meiosis. In Proceedings of the National Academy of Sciences of the United States of America, pp. 3951-3955.

1. Method for the identification of fungicides, characterised in that
(a) a host cell that expresses a thymidylate kinase in sufficient quantities or a polypeptide with the enzymatic activity of a thymidylate kinase is exposed to a chemical compound or a mixture of chemical compounds under conditions that allow interaction of the chemical compound with the polypeptide,
(b) the enzymatic activity of the thymidylate kinase in the absence of a chemical compound is compared with the enzymatic activity of the thymidylate kinase in the presence of a chemical compound or a mixture of chemical compounds, and
(c) the chemical compound is identified, which specifically inhibits the thymidylate kinase.

2. Method according to claim 1, characterised in that
(a) ADP occurring in the reaction of the thymidylate kinase is converted to ATP with the aid of a pyruvate kinase,
(b) the pyruvate occurring in the reaction is converted to lactate with the aid of a lactate dehydrogenase under consumption of NADH, and
(c) the consumption of NADH is tracked.

3. Method according to claim 2, characterised in that inhibition of enzymatic activity is determined on the basis of a decreased gain in ADP concentration.

4. Method according to one of claims 1 to 3, characterised in that the fungicidal activity of the identified compound is tested in a further step by exposing a fungus to the compound.

5. Method according to one of claims 1 to 4, characterised in that a thymidylate kinase is used from S. cerevisiae.

6. Method according to one of claims 1 to 4, characterised in that a thymidylate kinase is used from a plant pathogenic fungus.

7. Use of polypeptides with the activity of a thymidylate kinase for the identification of fungicides.

8. Use of inhibitors of fungal polypeptides with the activity of a thymidylate kinase as fungicides.

9. Use of inhibitors of a polypeptide with the activity of a thymidylate kinase, which were identified by a method according to one of claims 1 to 4, as fungicides.

10. Method for combating plant pathogenic fungi, characterised in that inhibitors of fungal polypeptides with the activity of a thymidylate kinase are allowed to interact with the plant pathogenic fungi and/or their habitat.
